(19) United States
(12) Patent Application Publication
(10) Pub. No.: US 2026/0115362 A1
(43) Pub. Date: Apr. 30, 2026

(54) BREAST PUMP

(71) Applicant: Shenzhen Oceanwing Smart Innovations Technology Co., Ltd, SHENZHEN (CN)

(72) Inventors: Liying CHEN, SHENZHEN (CN); Zhihai ZHANG, SHENZHEN (CN)

(73) Assignee: Shenzhen Oceanwing Smart Innovations Technology Co., Ltd, SHENZHEN (CN)

(21) Appl. No.: 19/365,238

(22) Filed: Oct. 22, 2025

(30) Foreign Application Priority Data
Oct. 25, 2024 (CN) 202422610405.9

(51) Int. Cl. A61M 1/06; A61M 39/00; A61M 39/24

(52) U.S. Cl. CPC A61M 1/064; A61M 39/24; A61M 2039/0009; A61M 2202/0014; A61M 2202/04; A61M 2205/3344; A61M 2205/3368; A61M 2205/36; A61M 2205/50; A61M 2205/82; A61M 2210/1007

(57) ABSTRACT

A breast pump, including a housing, a breast shield, and a main unit. The housing includes a first region and a second region. An orthogonal projection of the first region on the breast shield does not coincide with an orthogonal projection of the second region on the breast shield. The breast shield and the first region of the housing enclose to form a liquid storage chamber and a horn-shaped cavity. The liquid storage chamber is configured to store collected breast milk. The horn-shaped cavity is configured to accommodate a breast. A functional component is arranged within the breast shield. The main unit is disposed on a side of the housing away from the breast shield. The main unit is electrically connected to the functional component through an electrical conductor carrier. The electrical conductor carrier passes through the second region of the housing.

### CROSS-REFERENCE TO RELATED APPLICATIONS

The present application claims priority of Chinese Patent Application No. 202422610405.9, filed on Oct. 25, 2024, the entire contents of which are hereby incorporated by reference in their entirety.

### TECHNICAL FIELD

The present disclosure relates to the technical field of maternal and infant products, and specifically to a breast pump.

### BACKGROUND

A breast pump is an instrument used by postpartum women. When it is inconvenient to feed the infant, lactating women may apply the breast pump to extract breast milk from the breasts for storage and future use. This helps avoid waste of breast milk and facilitates the work and daily life of lactating women, which makes breast pumps widely popular in the market. With technological advancements, people have higher expectations for breast pumps. Some breast pumps with auxiliary functions, such as heated breast shields, have gradually appeared on the market. This kind of breast pump imposes higher requirements on the sealing performance of its liquid storage chamber.

### SUMMARY OF THE DISCLOSURE

A breast pump, including:
a housing, including a first region and a second region;
a breast shield, disposed on a side of the housing; wherein a projection of the first region along a thickness direction of the breast pump on the breast shield does not coincide with a projection of the second region along the thickness direction of the breast pump on the breast shield; the breast shield and the first region of the housing enclose to form a liquid storage chamber; a functional component is arranged within the breast shield; and
a main unit, disposed on another side of the housing away from the breast shield; wherein the main unit is electrically connected to the functional component through an electrical conductor carrier; the electrical conductor carrier passes through the second region of the housing.

A breast pump, including:
a housing, including a first region and a second region;
a breast shield, disposed on a side of the housing; wherein a projection of the first region along a thickness direction of the breast pump on the breast shield does not coincide with a projection of the second region along the thickness direction of the breast pump on the breast shield; the breast shield and the first region of the housing enclose to form a liquid storage chamber and a horn-shaped cavity, the liquid storage chamber being configured to store collected breast milk, and the horn-shaped cavity being configured to accommodate a breast; wherein a functional component is disposed within the breast shield; and
a main unit, disposed on another side of the housing away from the breast shield; wherein the main unit is electrically connected to the functional component through an electrical conductor carrier; the electrical conductor carrier passes through the second region of the housing.

A breast pump, including:
a housing;
a breast shield, disposed on a side of the housing; wherein the breast shield and the housing enclose to form a liquid storage chamber and a horn-shaped cavity; a functional component is disposed within the breast shield, and the functional component includes a heating element and a temperature sensor; and
a main unit, disposed on a side of the housing opposite to the breast shield; wherein the main unit includes a main control board, and the main control board integrates a negative pressure unit and a functional control unit; the negative pressure unit is configured to form negative pressure within the horn-shaped cavity, and the functional control unit is configured to be electrically connected to the functional component via a conductive carrier and to adjust a heating power of the heating element based on a signal from the temperature sensor.

### BRIEF DESCRIPTION OF THE DRAWINGS

To more clearly illustrate the technical solutions in the embodiments of the present disclosure, the accompanying drawings used in the description of the embodiments will be briefly introduced below. Obviously, the drawings in the following description are merely some embodiments of the present disclosure. For those skilled in the art, other drawings may be derived from these accompanying drawings without creative effort.

FIG. 1 is an overall structural schematic view of a breast pump according to some embodiments of the present disclosure.

FIG. 2 is a cross-sectional schematic view of the breast pump shown in FIG. 1.

FIG. 3 is an exploded schematic view of the breast pump shown in FIG. 1.

FIG. 4 is an exploded schematic view of the breast pump shown in FIG. 1 from another perspective.

FIG. 5 is an exploded schematic view of a breast shield shown in FIG. 1.

FIG. 6 is a schematic view of the connection relationship between a first contact and a second contact shown in FIG. 1.

FIG. 7 is a schematic view of the connection relationship between a first contact and a second contact according to other embodiments.

FIG. 8 is a cross-sectional schematic view of a housing shown in FIG. 1.

FIG. 9 is a schematic view of the cooperation relationship between the housing, a main unit, and a breast shield shown in FIG. 1.

FIG. 10 is a block diagram of a functional component according to some embodiments of the present disclosure.

FIG. 11 is a block diagram of a negative pressure unit and a functional control unit according to some embodiments of the present disclosure.

### DETAILED DESCRIPTION

The following describes the present disclosure in further detail with reference to the accompanying drawings and embodiments. It is noted that the following embodiments are only intended to illustrate the present disclosure but do not limit the scope of the present disclosure. Similarly, the following embodiments are only part of the embodiments of the present disclosure rather than all embodiments. All other embodiments obtained by those skilled in the art without creative effort shall fall within the scope of the present disclosure.

The terms “first”, “second”, and “third” in the embodiments of the present disclosure are intended for descriptive purposes only and cannot be understood as indicating or implying relative importance or implicitly specifying the quantity of the indicated technical features. Thus, features defined with “first”, “second”, and “third” may explicitly or implicitly include at least one such feature. In the description of the present disclosure, “a plurality of” means at least two, such as two, three, etc., unless otherwise specifically defined. All directional indications (such as up, down, left, right, front, back . . . ) in the embodiments of the present disclosure are only intended to explain relative positional relationships and movement conditions between various modules under a specific posture (as shown in the drawings). When the specific posture changes, the directional indications change accordingly. The terms “include”, “have” and any variations thereof in the embodiments of the present disclosure are intended to cover non-exclusive inclusion. For example, a process, method, system, product, or device that includes a series of steps or units is not limited to the listed steps or units, but may optionally further include steps or units not listed, or optionally include other steps or components inherent to these processes, methods, products, or devices.

Mentioning “embodiments” in this text means that the specific features, structures, or characteristics described in connection with the embodiments may be included in at least one embodiment of the present disclosure. The appearance of this phrase in various places in the specification does not necessarily refer to the same embodiment, nor is it an independent or alternative embodiment mutually exclusive with other embodiments. Those skilled in the art explicitly and implicitly understand that the embodiments described herein may be combined with other embodiments.

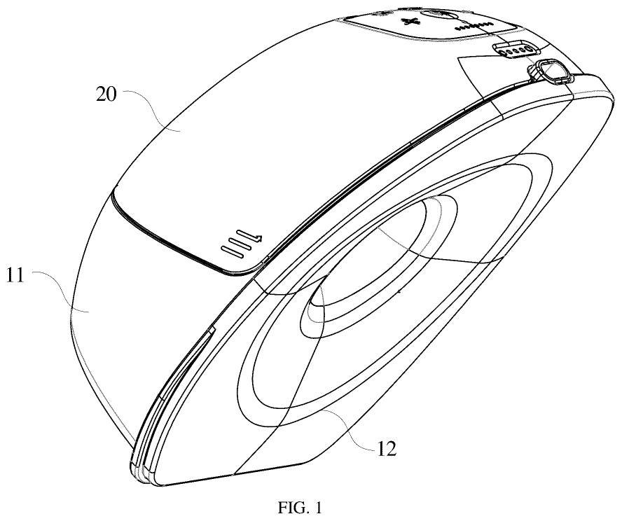

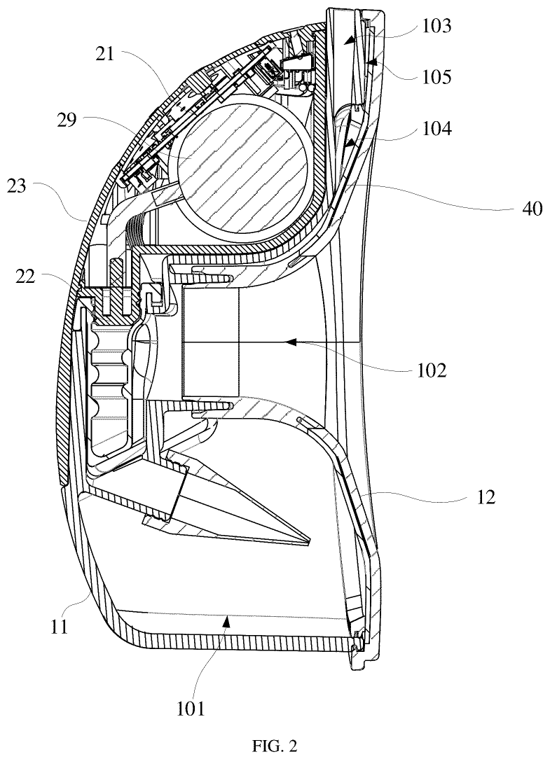

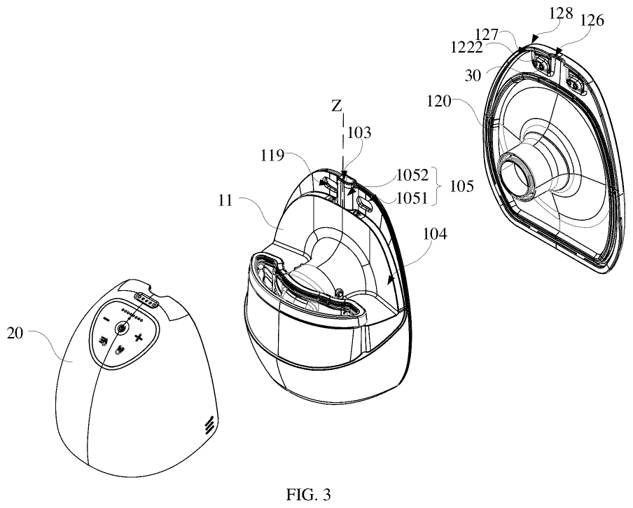

Referring to FIG. 1, FIG. 2, and FIG. 3, FIG. 1 is an overall structural schematic view of a breast pump according to some embodiments of the present disclosure, FIG. 2 is a cross-sectional schematic view of the breast pump shown in FIG. 1, and FIG. 3 is an exploded schematic view of the breast pump shown in FIG. 1.

Embodiments of the present disclosure provide a breast pump, including a housing 11, a breast shield 12, and a main unit 20. The housing 11 includes a first region 104 and a second region 105. An orthogonal projection of the first region 104 on the breast shield 12 does not coincide with an orthogonal projection of the second region 105 on the breast shield 12. The breast shield 12 and the first region 104 of the housing 11 enclose to form a liquid storage chamber 101 and a horn-shaped cavity 102. The liquid storage chamber 101 is configured to store collected breast milk. The horn-shaped cavity 102 is configured to accommodate a breast. A functional component 40 is arranged within the breast shield 12. The main unit 20 is disposed on a side of the housing 11 away from the breast shield 12. The main unit 20 is electrically connected to the functional component 40 through an electrical conductor carrier 30. The electrical conductor carrier 30 passes through the second region 105 of the housing 11.

The functional component 40 may specifically be a component that implements any single function such as applying heat or massaging the breast, or simultaneously implements multiple functions. Referring to FIG. 11, the main unit 20 includes a negative pressure unit 21A and a functional control unit 21B. An output end of the negative pressure unit 21A is in communication with the horn-shaped cavity 102 and is configured to control the formation of negative pressure within the horn-shaped cavity 102. The functional control unit 21B is electrically connected to the functional component 40 through the electrical conductor carrier 30. The electrical conductor carrier 30 is disposed outside the liquid storage chamber 101 and the horn-shaped cavity 102.

By virtue of the above solutions, the liquid storage chamber 101 is formed by the enclosure of the breast shield 12 and the housing 11, thereby optimizing the space of the liquid storage chamber 101, making the structure more compact, and increasing the liquid storage capacity. By dividing the housing 11 into the first region 104 and the second region 105 whose orthogonal projections on the breast shield 12 do not coincide, the liquid storage chamber 101 and the electrical conductor carrier 30 are disposed in the first region 104 and the second region 105, respectively, thereby improving the sealing performance of the breast pump and preventing short circuits or poor contact caused by the electrical conductor carrier 30 being immersed in the breast milk.

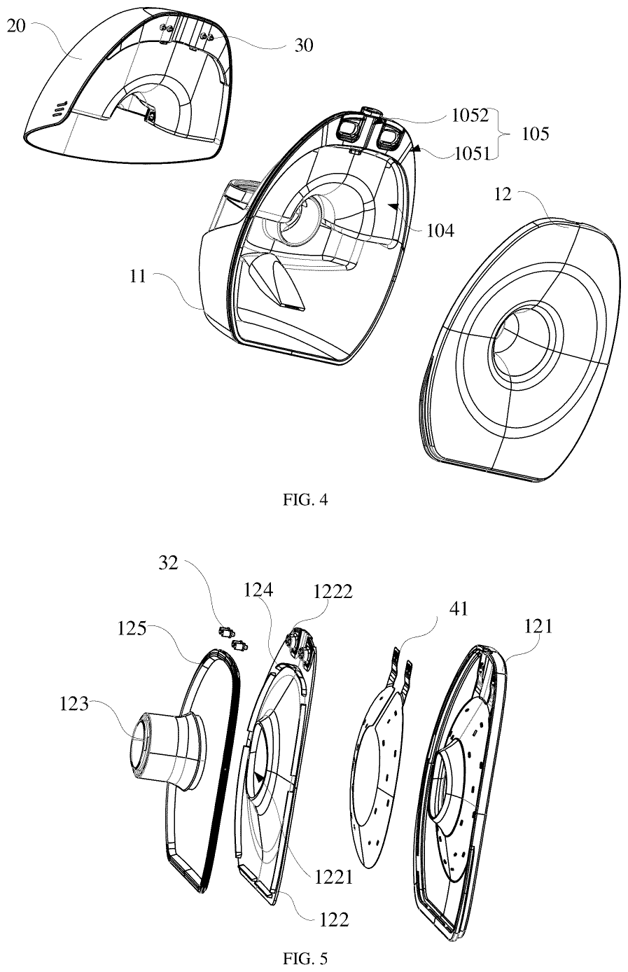

Referring to FIG. 3 and FIG. 4, FIG. 4 is an exploded schematic view of the breast pump shown in FIG. 1 from another perspective. In some embodiments, the second region 105 includes a connection region 1051 and a liquid outlet region 1052. An orthogonal projection of the connection region 1051 on the breast shield 12 does not coincide with an orthogonal projection of the liquid outlet region 1052 on the breast shield 12. The electrical conductor carrier 30 passes through the connection region 1051. The liquid outlet region 1052 defines a liquid pouring channel 103. The liquid pouring channel 103 is in communication with the liquid storage chamber 101.

In some embodiments, the liquid outlet region 1052 is a closed annular structure. The liquid outlet region 1052 encloses to form the liquid pouring channel 103 to isolate the liquid pouring channel 103 from the connection region 1051. That is, the liquid pouring channel 103 is defined by an inner wall of this annular structure. An end of the liquid pouring channel 103 is in communication with the liquid storage chamber 101, and the other end of the liquid pouring channel 103 extends to an edge of the housing 11. During the process of pouring out the breast milk, the breast milk can flow directly from the first region 104 along the liquid pouring channel 103 through the second region 105. The inner and outer walls of the annular structure are not interconnected. Therefore, when the breast milk flows through the second region 105, it is confined to flow within the liquid outlet region 1052, thereby preventing the breast milk from contacting the electrical conductor carrier 30 in the connection region 1051. The annular structure may specifically have a cross-section that is circular, square, or other closed special shapes, which is not specifically limited herein.

In some embodiments, the liquid outlet region 1052 is an open trough structure. The liquid pouring channel 103 is defined by a trough wall of this trough structure. An end of the liquid pouring channel 103 is in communication with the liquid storage chamber 101, and the other end of the liquid pouring channel 103 extends to an edge of the housing 11. During the process of pouring out the breast milk, an opening of the trough structure may be facing upward, and the breast milk can be poured out slowly along the trough wall.

Referring to FIG. 3, in some embodiments, the liquid outlet region 1052 is disposed at a central axis position of the housing 11. The dashed line Z shown in FIG. 3 is the central axis of the housing 11. The connection region 1051 is symmetrically distributed on both sides of the central axis. An extension direction of the liquid pouring channel 103 is consistent with an extension direction of the central axis. During use, the liquid pouring channel 103 may be placed vertically upward to prevent the breast milk from flowing out under gravity. Of course, a plug or solenoid valve may further be arranged to block the liquid pouring channel 103.

Continuing to refer to FIG. 3, in some embodiments, the breast shield 12 includes an inner side 127 and an outer side 128 that are opposite to each other. An avoidance groove 126 is defined on the breast shield 12, penetrating the inner side 127 and the outer side 128 of the breast shield 12. An opening of the avoidance groove 126 matches part of an outer contour of the liquid outlet region 1052. The connection region 1051 is adjacent to the inner side 127 of the breast shield 12. The liquid outlet region 1052 passes through the avoidance groove 126 and extends to the outer side 128 of the breast shield 12. That is, the liquid outlet region 1052 protrudes relative to the connection region 1051. This allows a protruding part of the liquid outlet region 1052 to match with a mouth of a milk storage bottle, facilitating the pouring of the extracted breast milk into the milk storage bottle for storage.

Combined with the implementation style where the liquid outlet region 1052 is an annular structure, the main unit 20 may also define a groove at a position corresponding to the liquid outlet region 1052. The groove on the main unit 20 and the avoidance groove 126 on the breast shield 12 combine to form a ring matching the outer contour shape of the annular structure, allowing the liquid outlet region 1052 to extend to the outer sides of the main unit 20 and the breast shield 12.

Referring to FIG. 3, the breast shield 12 may include a sealing member 120. The sealing member 120 is disposed on a side of the breast shield 12 facing the housing 11. The sealing member 120 divides the housing 11 into the first region 104 and the second region 105. It should be noted that, combined with the implementation style where the second region 105 includes the connection region 1051 and the liquid outlet region 1052 and the liquid outlet region 1052 is a closed annular structure, the first region 104 and the connection region 1051 are isolated by the sealing member 120, and the connection region 1051 and the liquid outlet region 1052 are isolated by the annular structure formed by the housing 11 itself. This ensures that the connection region 1051 is always isolated from the first region 104 and the liquid outlet region 1052 during the operation of the breast pump and during liquid pouring, thereby preventing short circuits or poor contact caused by the breast milk infiltrating the electrical conductor carrier 30.

Referring to FIG. 5, FIG. 5 is an exploded schematic view of a breast shield shown in FIG. 1. Specifically, the sealing member 120 may include a rib 124. The rib 124 protrudes from the side of the breast shield 12 facing the housing 11 towards the housing 11. The rib 124 is fixedly attached to an inner wall of the housing 11 to form the liquid storage chamber 101.

In some embodiments, the sealing member 120 further includes a flexible sealing ring 125 sleeved on the rib 124. When the breast shield 12 is assembled and connected to the housing 11, the flexible sealing ring 125 is squeezed into a gap between the rib 124 and the inner wall of the housing 11, forming an expansion force to ensure the sealing of the liquid storage chamber 101 and the stability of the connection between the breast shield 12 and the housing 11.

In some embodiments, the sealing member 120 may be disposed on a side of the housing 11 facing the breast shield 12. The side of the breast shield 12 facing the housing 11 defines a groove matching the shape of the first region 104. The sealing member 120 is embedded in the groove to achieve sealed attachment between the housing 11 and the breast shield 12.

In some embodiments, the liquid outlet region 1052 may be disposed in the first region 104. For example, the liquid outlet region 1052 is disposed on an end of the first region 104 away from the second region 105. The side of the breast shield 12 facing the housing 11 is arranged with the sealing member 120, and the first region 104 and the second region 105 are isolated by the sealing member 120. The liquid outlet region 1052 defines the liquid pouring channel 103, which is in communication with the liquid storage chamber 101. When the breast pump is working, the liquid pouring channel 103 may be placed vertically upward, then the second region 105 is disposed below the first region 104. Since the first region 104 and the second region 105 are isolated by the sealing member 120, the breast milk may be prevented from flowing from the liquid storage chamber 101 in the first region 104 to the second region 105, thereby damaging the electrical conductor carrier 30 in the second region 105. During liquid pouring, the liquid pouring channel 103 may be facing downward, then the first region 104 is disposed below the second region 105, and the liquid outlet region 1052 is disposed on the end of the first region 104 away from the second region 105. This may prevent the breast milk from spreading to the second region 105 when it flows through the liquid outlet region 1052.

Referring to FIG. 5, in some embodiments, the breast shield 12 includes a liquid outlet pipe 123, a first shield body 121, and a second shield body 122, where an outer diameter of the first shield body 121 and an outer diameter of the second shield body 122 each gradually increase in a direction away from the liquid outlet pipe 123. The second shield body 122 defines a mounting hole 1221 matching the shape of the liquid outlet pipe 123. The second shield body 122 is sleeved on the liquid outlet pipe 123 through the mounting hole 1221 and is attached to a side of the first shield body 121 facing the liquid outlet pipe 123. The functional component 40 is embedded between the first shield body 121 and the second shield body 122. The second shield body 122 may be made of hard plastic to serve as a support body. The first shield body 121 may be made of soft plastic to better fit the breast. The first shield body 121 and the second shield body 122 may be integrally formed and combined together. The hard plastic may be TRITAN, PPSU, or PP, etc. The soft plastic may be silicone, TPE, TPU, or other elastic materials.

Furthermore, the sealing member 120 is disposed on the second shield body 122. The flexible sealing ring 125 is connected to the rib 124 and surrounds to form a closed area. A side of the second shield body 122 facing the housing 11 is arranged with a positioning post 1222. The positioning post 1222 is disposed outside the closed area formed by the flexible sealing ring 125, specifically in the connection region 1051. The housing 11 defines a positioning hole 119 at a position corresponding to the positioning post 1222. The positioning post 1222 is inserted into the positioning hole 119 to facilitate the assembly and fixation of the housing 11 and the breast shield 12.

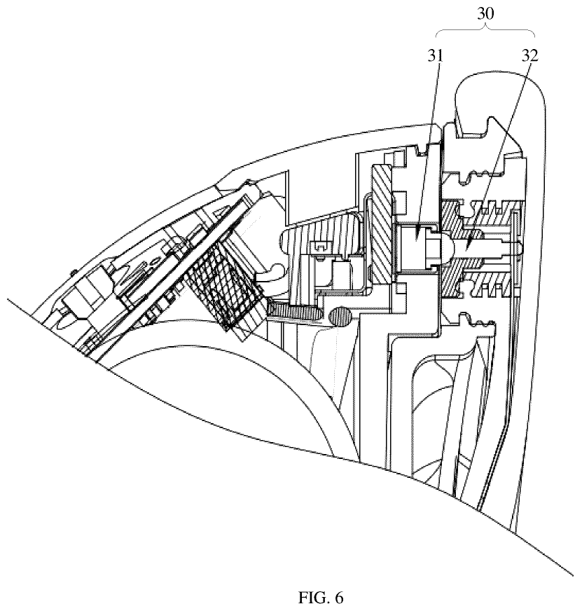

Referring to FIG. 6, FIG. 6 is a schematic view of the connection relationship between a first contact and a second contact shown in FIG. 1. Further, an installation cavity is defined within the positioning post 1222. The electrical conductor carrier 30 includes a first contact 31 disposed on the main unit 20 and electrically connected to the functional control unit 21B, and a second contact 32 disposed on the breast shield 12 and electrically connected to the functional component 40. The first contact 31 is inserted into the installation cavity and is in contact-type electrical connection with the second contact 32 to enable the main unit 20 to supply power to the functional component 40. Since the positioning post 1222 is disposed in the connection region 1051, the positioning post 1222 is disposed outside the liquid storage chamber 101 formed by the enclosure of the breast shield 12 and the housing 11. That is, the connection position of the first contact 31 and the second contact 32 is disposed outside the liquid storage chamber 101, thereby preventing damage caused by the first contact 31 and the second contact 32 being immersed in the breast milk.

In other embodiments, the positioning post 1222 may be disposed on a side of the main unit 20 facing the breast shield 12. An installation cavity is defined within the positioning post 1222. The second contact 32 is disposed in the installation cavity. The first contact 31 is inserted into the installation cavity and is in contact-type electrical connection with the second contact 32, thereby enabling the main unit 20 to supply power to the functional component 40.

In some embodiments, the first contact 31 is a rigid contact, and the second contact 32 is an elastic contact; in some embodiments, the first contact 31 is an elastic contact, and the second contact 32 is a rigid contact; in some embodiments, both the first contact 31 and the second contact 32 are rigid contacts; in some embodiments, both the first contact 31 and the second contact 32 are elastic contacts.

Continuing to refer to FIG. 6, in some embodiments, extension directions of the first contact 31 and the second contact 32 are coaxial. In the embodiments, the installation cavity may be a cavity that passes through the positioning post 1222 along its axial direction. The first contact 31 and the second contact 32 are inserted into the installation cavity from opposite sides of the positioning post 1222 respectively and contact each other.

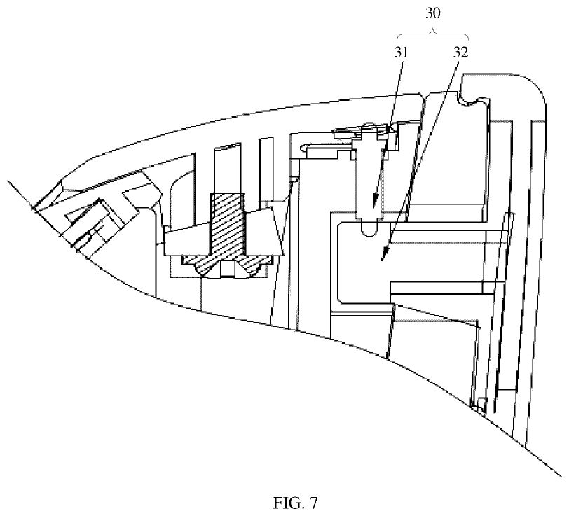

Referring to FIG. 7, FIG. 7 is a schematic view of the connection relationship between a first contact and a second contact according to other embodiments. In some embodiments, the extension directions of the first contact 31 and the second contact 32 intersect. For example, the extension directions of the first contact 31 and the second contact 32 are orthogonal. In the embodiments, the first contact 31 may be embedded in the installation cavity from an annular side of the positioning post 1222 and contact the second contact 32 in the installation cavity. A peripheral side of the first contact 31 is tightly attached to a side wall of the installation cavity, meaning the positioning post 1222 plays a limiting role for the first contact 31, thereby improving the stability of the connection between the main unit 20 and the breast shield 12.

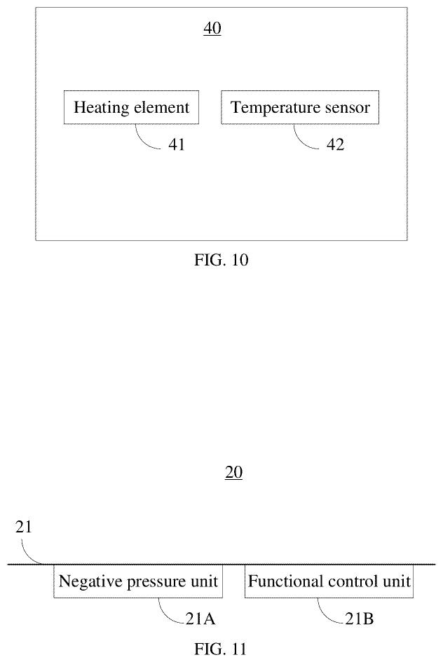

Referring again to FIGS. 5 and 10, in some embodiments, the functional component 40 includes a heating element 41 and a temperature sensor 42. Four first contacts 31 are provided, corresponding to and connected to positive and negative poles of the heating element 41 and positive and negative poles of the temperature sensor 42, respectively. Similarly, four second contacts 32 are provided, corresponding to and electrically connected to the four first contacts 31 one by one.

The heating element 41 is sheet-like and matches the shape of the breast shield 12, allowing the breast shield 12 to be heated evenly, thereby making the temperature of the contact surface between the breast shield 12 and the breast uniform. The heating element 41 may define multiple through holes. A side of the first shield body 121 and a side of the second shield body 122 are arranged with concave-convex structures, where the side of the first shield body 121 and the side of the second shield body 122 face each other. For example, the side of the first shield body 121 is arranged with protrusions, and the side of the second shield body 122 defines recesses. When assembling the heating element 41, the protrusions on the first shield body 121 can pass through the through holes on the heating element 41 and embed into the recesses on the second shield body 122, which may ensure the stability of the assembly connection of the heating element 41 between the first shield body 121 and the second shield body 122, thereby preventing the heating element 41 from shifting during use.

The functional component 40 may further include devices for other functions such as massage. For example, the functional component 40 further includes a vibrator. The vibrator is also embedded between the first shield body 121 and the second shield body 122 and is electrically connected to the functional control unit 21B of the main unit 20 through the electrical conductor carrier 30. After the vibrator and the functional control unit 21B form an energized circuit, vibration is generated, which can massage the breast and promote breast milk secretion.

Referring to FIG. 2, the functional control unit 21B and the negative pressure unit 21A may be integrated on the same main control board 21. The main unit 20 may further include a battery. The battery is connected to the main control board 21 and can provide working power for the breast pump. Of course, the breast pump may be powered by an external power source.

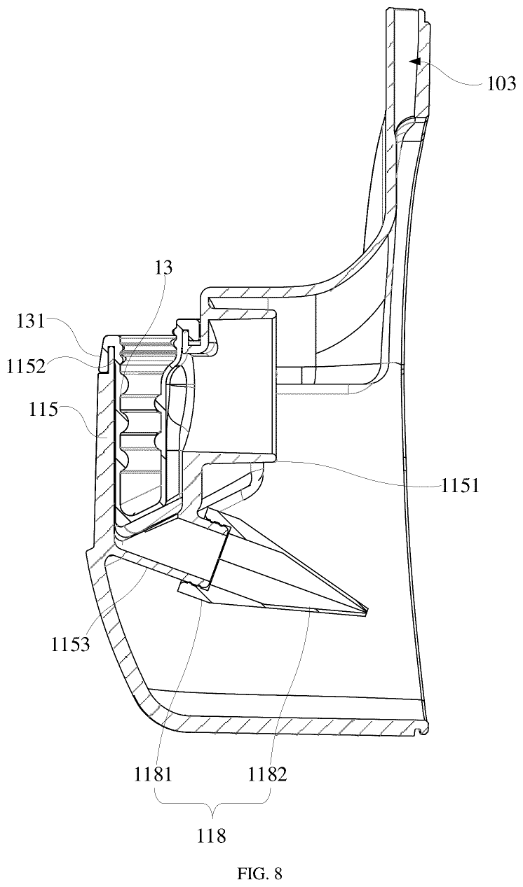

Referring to FIG. 8, FIG. 8 is a cross-sectional schematic view of a housing shown in FIG. 1. In some embodiments, a first accommodation portion 115 is arranged inside the housing 11. The first accommodation portion 115 is connected to the negative pressure unit 21A, and a deformable sealing member 13 is arranged between the first accommodation portion 115 and the negative pressure unit 21A. At least part of the horn-shaped cavity 102 is defined by an inner wall of the first accommodation portion 115. The deformable sealing member 13 is configured to deform under the action of the negative pressure unit 21A, thereby creating negative pressure within the horn-shaped cavity 102.

Specifically, the first accommodation portion 115 is connected to the liquid outlet pipe 123 through a liquid inlet pipe 1151. That is, the horn-shaped cavity 102 is enclosed by the breast shield 12, the liquid outlet pipe 123, the liquid inlet pipe 1151, the first accommodation portion 115, and the deformable sealing member 13. The deformable sealing member 13 can deform under the action of the negative pressure unit 21A, thereby changing the space size of the horn-shaped cavity 102, creating negative pressure within the horn-shaped cavity 102, and thus extracting the breast milk from the breast.

Referring to FIG. 2 and FIG. 8, the first accommodation portion 115 is arranged with a negative pressure port 1152. The main unit 20 is arranged with a negative pressure connector 22. The negative pressure connector 22 can be connected to the negative pressure port 1152 and communicate with the deformable cavity. The negative pressure unit 21A includes a suction pump 29. The suction pump 29 is connected to the negative pressure connector 22 through a pipeline. The deformable sealing member 13 may be disposed inside or outside the first accommodation portion 115.

For an implementation where the deformable sealing member 13 is disposed inside the first accommodation portion 115, the deformable sealing member 13 has an opening. The opening of the deformable sealing member 13 is sleeved on the negative pressure port 1152 and defines a deformable cavity inside the first accommodation portion 115. During the negative pressure output of the suction pump 29, the suction pump 29 sucks air from the deformable sealed cavity, causing the deformable sealing member 13 to contract, thereby defining a negative pressure space between an outer wall of the deformable sealing member 13 and an inner wall of the first accommodation portion 115. The negative pressure in the negative pressure space acts on the breast through the liquid inlet pipe 1151 and the liquid outlet pipe 123, causing the breast to secrete the breast milk. In the embodiments, the negative pressure port 1152 can extend outward from the first accommodation portion 115 for a certain distance. The opening of the deformable sealing member 13 has a first flange 131 that is flanged outward. The first flange 131 is sleeved on an end of the negative pressure port 1152. The negative pressure connector 22 is inserted into the negative pressure port 1152. An end of the negative pressure connector 22 presses the first flange 131 against the end of the negative pressure port 1152. Thus, the first flange 131 is clamped between the negative pressure connector 22 and the negative pressure port 1152, ensuring the installation airtightness and stability of the deformable sealing member 13. Since the deformable sealing member 13 is disposed inside the first accommodation portion 115 and shares the same negative pressure space with the breast milk, the space occupied by the breast pump may be reduced, allowing the breast pump to be designed smaller for portability. In addition, the deformable sealing member 13 is disposed between the negative pressure port 1152 and the negative pressure connector 22, thereby preventing short circuits or damage to the electronic components inside caused by the breast milk entering the main unit 20.

For an implementation where the deformable sealing member 13 is disposed outside the first accommodation portion 115, the deformable sealing member 13 is sleeved on the negative pressure connector 22 and defines a deformable sealed cavity within the negative pressure connector 22. The suction pump 29 can suck air from a space defined between an outer wall of the deformable sealing member 13 and an inner wall of the negative pressure connector 22, causing the deformable sealing member 13 to expand, thereby defining a negative pressure space between an inner wall of the deformable sealing member 13 and an inner wall of the first accommodation portion 115. The negative pressure in the negative pressure space acts on the breast through the liquid inlet pipe 1151 and the liquid outlet pipe 123, causing the breast to secrete the breast milk.

Continuing to refer to FIG. 8, in some embodiments, a check valve 118 is arranged between the liquid storage chamber 101 and the horn-shaped cavity 102. The check valve 118 allows liquid in the horn-shaped cavity 102 to flow into the liquid storage chamber 101 and prevents liquid in the liquid storage chamber 101 from flowing back into the horn-shaped cavity 102. For example, a liquid discharge pipe 1153 extends from the first accommodation portion 115 towards the liquid storage chamber 101. The liquid discharge pipe 1153 is integrally formed with the housing 11. The liquid discharge pipe 1153 may be made of hard plastic. The check valve 118 may be made of soft plastic. The check valve 118 is detachably sleeved on the liquid discharge pipe 1153. The shape of the check valve 118 may be arbitrary, such as vertical or flat. In a case where the check valve 118 is flat, the check valve 118 may include a connection portion 1181 and a suction portion 1182. The connection portion 1181 is nested on an outside of the liquid discharge pipe 1153. The suction portion 1182 communicates with the liquid discharge pipe 1153 through the connection portion 1181. During the negative pressure output of the suction pump 29, the air pressure output by the suction pump 29 causes the deformable sealing member 13 to deform, thereby reducing the air pressure inside the first accommodation portion 115 to form negative pressure, and thus causing the check valve 118 to suction. The formed negative pressure acts on the breast through the liquid inlet pipe 1151 and the liquid outlet pipe 123. When the suction pump 29 stops outputting negative pressure, the shape of the deformable sealing member 13 recovers, and the air pressure inside the first accommodation portion 115 recovers accordingly. Consequently, the check valve 118 is no longer in the suction state. The breast milk flows into the liquid storage chamber 101 through the liquid discharge pipe 1153 and the check valve 118 under gravity.

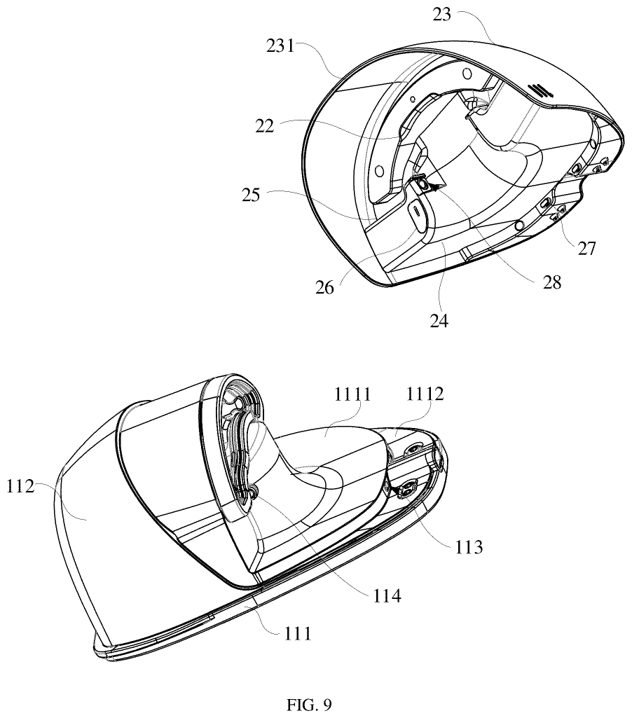

Referring to FIG. 9, FIG. 9 is a schematic view of the cooperation relationship between the housing, a main unit, and a breast shield shown in FIG. 1. In some embodiments, the housing 11 includes a support portion 111 and a protrusion portion 112. The support portion 111 is connected to the breast shield 12. The protrusion portion 112 protrudes from the support portion 111 in a direction away from the breast shield 12. The main unit 20 is assembled on the support portion 111 and connected to the protrusion portion 112. The breast shield 12 is assembled on the support portion 111, and the second contact 32 on the breast shield 12 is in contact-type electrical connection with the first contact 31 on the main unit 20, thereby forming a structurally stable combination. For example, the connection between the main unit 20 and the protrusion portion 112 has a smooth transition, forming a surface that is flush or substantially flush, either planar or curved. A peripheral surface of the main unit 20 and a peripheral surface of the protrusion portion 112 may be both curved surfaces. The support portion 111 may have an elliptical contour. The assembled main unit 20 and housing 11 form a semi-ellipsoid or hemisphere.

The main unit 20 includes a main unit housing 23, a first cover plate 24, and a second cover plate 25, where the first cover plate 24 and the second cover plate 25 are disposed at an angle. The first cover plate 24, the second cover plate 25, and the main unit housing 23 enclose to define a sealed main unit space. The angle defined by the first cover plate 24 and the second cover plate 25 matches an angle defined by the protrusion portion 112 and the support portion 111. The first cover plate 24 may be attached to a side of the support portion 111 away from the breast shield 12. The second cover plate 25 may be attached to an outer surface of the protrusion portion 112.

A mating surface of the support portion 111 with the first cover plate 24 and the breast shield 12 is stepped. For example, the support portion 111 includes a first step 1111 and a second step 1112. The second step 1112 extends outward from the first step 1111. When the breast shield 12 is connected to the support portion 111, the sealing member 120 on the breast shield 12 may be fixedly attached to an inner ring side surface of the first step 1111 to form the sealed liquid storage chamber 101 between the breast shield 12 and the housing 11. In addition, the second step 1112 of the support portion 111 forms an assembly area that does not coincide with the liquid storage chamber 101, i.e., the second region 105. The first contact 31 and the second contact 32 achieve contact-type electrical connection in this assembly area.

A magnetic attraction member 26 is arranged between the second cover plate 25 and the protrusion portion 112 for attracting and fixing the second cover plate 25 and the protrusion portion 112. The negative pressure connector 22 is disposed on the second cover plate 25. The negative pressure connector 22 is pressed into the negative pressure port 1152. A magnetic force direction of the magnetic attraction member 26 is the same as an extension direction of the negative pressure connector 22 and the negative pressure port 1152 to further ensure the reliability of the connection between the negative pressure connector 22 and the negative pressure port 1152. The first contact 31 is disposed on the first cover plate 24. The first contact 31 may be snapped into the positioning post 1222 and contact the second contact 32.

In addition, the main unit housing 23 has an outer edge portion 231 extending beyond the second cover plate 25. The outer edge portion 231 overlaps the protrusion portion 112. The outer edge portion 231 is tangent to the outer surface of the protrusion portion 112, achieving a smooth transition between the main unit housing 23 and the protrusion portion 112.

An engagement structure is arranged between mating surfaces of the first cover plate 24 and the support portion 111, and/or between mating surfaces of the second cover plate 25 and the protrusion portion 112. For example, the first cover plate 24 is arranged with a first engaging block 27. The first step 1111 of the support portion 111 defines a first slot 113 matching the first engaging block 27. The second cover plate 25 is arranged with a second slot 28. The protrusion portion 112 defines a second engaging block 114 matching the second slot 28. The extension directions of the first slot 113 and the second slot 28 are not parallel. The engagement structure formed by the first slot 113 and the first engaging block 27 and the engagement structure formed by the second slot 28 and the second engaging block 114 may restrict movement mutually, thereby facilitating a reliable connection between the main unit 20 and the housing 11.

In the embodiments, when the main unit 20 is assembled with the housing 11 and the breast shield 12, the outer edge portion 231 of the main unit housing 23 can cooperate with the outer surface of the protrusion portion 112, for guiding the negative pressure connector 22 to slide along the negative pressure port 1152 until the negative pressure connector 22 is inserted into the negative pressure port 1152 in an engaged state. In this case, the first cover plate 24 is attached to the support portion 111, the second cover plate 25 is attached to the protrusion portion 112, the first contact 31 is inserted into the positioning post 1222, the first engaging block 27 is inserted into the first slot 113, and the second engaging block 114 is inserted into the second slot 28, thereby achieving relative fixation between the main unit 20 and the housing 11 and preventing relative movement after installation.

The above is only some of the embodiments of the present disclosure and does not therefore limit the scope of the present disclosure. Any equivalent device or equivalent process transformation made using the specification and drawings of the present disclosure, or directly or indirectly applied in other related technical fields, shall similarly be included within the scope of the present disclosure.

1. A breast pump, comprising:
a housing, comprising a first region and a second region;
a breast shield, disposed on a side of the housing; wherein a projection of the first region along a thickness direction of the breast pump on the breast shield does not coincide with a projection of the second region along the thickness direction of the breast pump on the breast shield; the breast shield and the first region of the housing enclose to form a liquid storage chamber; a functional component is arranged within the breast shield; and
a main unit, disposed on another side of the housing away from the breast shield; wherein the main unit is electrically connected to the functional component through an electrical conductor carrier; the electrical conductor carrier passes through the second region of the housing.

2. The breast pump according to claim 1, wherein the second region comprises a connection region and a liquid outlet region; a projection of the connection region along the thickness direction of the breast pump on the breast shield does not coincide with a projection of the liquid outlet region along the thickness direction of the breast pump on the breast shield;
the liquid outlet region defines a liquid pouring channel for communicating the liquid storage chamber with an external environment; the electrical conductor carrier passes through the connection region.

3. The breast pump according to claim 2, wherein the liquid outlet region is disposed at a position of a central axis of the housing; the connection region is symmetrically distributed on both sides of the central axis.

4. The breast pump according to claim 2, wherein the liquid outlet region is a closed annular structure; the liquid pouring channel is defined by an inner wall of the annular structure to isolate the liquid pouring channel from the connection region;
an end of the liquid pouring channel is in communication with the liquid storage chamber, and another end of the liquid pouring channel extends to an edge of the housing.

5. The breast pump according to claim 4, wherein the breast shield comprises an inner side and an outer side that are opposite to each other; an avoidance groove is defined on the breast shield; the connection region is adjacent to the inner side of the breast shield; the liquid outlet region passes through the avoidance groove and extends to the outer side of the breast shield.

6. The breast pump according to claim 1, wherein the breast shield comprises a sealing member; the sealing member is disposed on a side of the breast shield facing the housing; the sealing member is sealingly fitted to an inner wall of the housing to form the liquid storage chamber, and the sealing member divides the housing into the first region and the second region.

7. The breast pump according to claim 6, wherein the sealing member comprises a rib; the rib protrudes from the side of the breast shield facing the housing towards the housing; the rib is fixedly attached to an inner wall of the housing to form the liquid storage chamber.

8. The breast pump according to claim 7, wherein the sealing member further comprises a flexible sealing ring sleeved on the rib; the flexible sealing ring is squeezed into a gap between the rib and the inner wall of the housing, forming an expansion force.

9. The breast pump according to claim 2, wherein a positioning post is arranged on a side of the breast shield facing the main unit or on a side of the main unit facing the breast shield; the housing defines a positioning hole at a position corresponding to the positioning post; the positioning hole is disposed on the connection region, and the positioning post is inserted into the positioning hole.

10. The breast pump according to claim 9, wherein an installation cavity is defined within the positioning post; the electrical conductor carrier comprises a first contact disposed on the main unit and a second contact disposed on the breast shield and electrically connected to the functional component;
the first contact is disposed in the installation cavity; the first contact is inserted into the installation cavity and is in contact-type electrical connection with the second contact to enable the main unit to supply power to the functional component; or
the second contact is disposed in the installation cavity; the first contact is inserted into the installation cavity and is in contact-type electrical connection with the second contact to enable the main unit to supply power to the functional component.

11. The breast pump according to claim 10, wherein the breast shield comprises a liquid outlet pipe, a first shield body, and a second shield body, where an outer diameter of the first shield body and an outer diameter of the second shield body each gradually increase in a direction away from the liquid outlet pipe;
the second shield body defines a mounting hole matching a shape of the liquid outlet pipe; the second shield body is sleeved on the liquid outlet pipe through the mounting hole and is attached to a side of the first shield body facing the liquid outlet pipe; the functional component is embedded between the first shield body and the second shield body; an end of the second contact is electrically connected to the functional component, and another end of the second contact extends to a surface of the second shield body.

12. The breast pump according to claim 11, wherein the functional component comprises a heating element; the heating element defines a plurality of through holes; one of a side of the first shield body and a side of the second shield body is arranged with protrusions, and the other of the side of the first shield body and the side of the second shield body defines recesses, where the side of the first shield body and the side of the second shield body face each other;
the protrusions are configured to pass through the plurality of through holes on the heating element and embed into the recesses, for fixing the heating element between the first shield body and the second shield body.

13. The breast pump according to claim 10, wherein an extension direction of the first contact and an extension direction of the second contact are coaxial; the installation cavity is a cavity that passes through the positioning post along an axial direction; the first contact and the second contact are inserted into the installation cavity from opposite sides of the positioning post respectively and contact each other.

14. The breast pump according to claim 10, wherein an extension direction of the first contact and an extension direction of the second contact are intersected; the first contact is embedded in the installation cavity from an annular side of the positioning post and contact the second contact in the installation cavity; a peripheral side of the first contact is tightly attached to a side wall of the installation cavity.

15. The breast pump according to claim 1, wherein the breast shield and the housing enclose to further form a horn-shaped cavity; the horn-shaped cavity is configured to accommodate a breast; a check valve is arranged between the liquid storage chamber and the horn-shaped cavity; the check valve is configured for liquid in the horn-shaped cavity to flow into the liquid storage chamber and prevent liquid in the liquid storage chamber from flowing back into the horn-shaped cavity.

16. The breast pump according to claim 1, wherein the housing comprises a support portion and a protrusion portion; the support portion is connected to the breast shield; the protrusion portion protrudes from the support portion in a direction away from the breast shield; the main unit is assembled on the support portion and connected to the protrusion portion; the breast shield is assembled on the support portion, and the second contact on the breast shield is in contact-type electrical connection with the first contact on the main unit.

17. The breast pump according to claim 16, wherein the main unit comprises a main unit housing, a first cover plate, and a second cover plate, where the first cover plate and the second cover plate are disposed at an angle; the first cover plate, the second cover plate, and the main unit housing enclose to define a sealed main unit space; The angle defined by the first cover plate and the second cover plate matches an angle defined by the protrusion portion and the support portion; the first cover plate is attached to a side of the support portion away from the breast shield; the second cover plate is attached to an outer surface of the protrusion portion.

18. The breast pump according to claim 17, wherein the support portion comprises a first step and a second step; the second step extends outward from the first step;
the breast shield comprises a sealing member; the sealing member is fixedly attached to an inner ring side surface of the first step to form the liquid storage chamber between the breast shield and the housing;
the electrical conductor carrier comprises a first contact disposed on the main unit and a second contact disposed on the breast shield and electrically connected to the functional component; the second step forms an assembly area that does not coincide with the liquid storage chamber; the first contact and the second contact achieve contact-type electrical connection in the assembly area.

19. A breast pump, comprising:
a housing, comprising a first region and a second region;
a breast shield, disposed on a side of the housing; wherein a projection of the first region along a thickness direction of the breast pump on the breast shield does not coincide with a projection of the second region along the thickness direction of the breast pump on the breast shield; the breast shield and the first region of the housing enclose to form a liquid storage chamber and a horn-shaped cavity, the liquid storage chamber being configured to store collected breast milk, and the horn-shaped cavity being configured to accommodate a breast; wherein a functional component is disposed within the breast shield; and
a main unit, disposed on another side of the housing away from the breast shield; wherein the main unit is electrically connected to the functional component through an electrical conductor carrier; the electrical conductor carrier passes through the second region of the housing.

20. A breast pump, comprising:
a housing;
a breast shield, disposed on a side of the housing; wherein the breast shield and the housing enclose to form a liquid storage chamber and a horn-shaped cavity; a functional component is disposed within the breast shield, and the functional component comprises a heating element and a temperature sensor; and
a main unit, disposed on a side of the housing opposite to the breast shield; wherein the main unit comprises a main control board, and the main control board integrates a negative pressure unit and a functional control unit; the negative pressure unit is configured to form negative pressure within the horn-shaped cavity, and the functional control unit is configured to be electrically connected to the functional component via a conductive carrier and to adjust a heating power of the heating element based on a signal from the temperature sensor.
